(19) United States
(12) Patent Application Publication
(10) Pub. No.: US 2015/0045432 A1
(43) Pub. Date: Feb. 12, 2015

(54) DIETARY SUPPLEMENT COMPRISING AMINO ACIDS IN A PALATABLE LIQUID FORMULATION THAT PROMOTES RESTFUL SLEEP, RECOVERY FROM STRESS AND EXERCISE AND STRENGTHENS THE IMMUNE SYSTEM

(71) Applicant: North Health Essentials, LLC, Wilmington, DE (US)

(72) Inventors: Robert North, Wilmington, DE (US); James Crute, Wilmington, DE (US)

(73) Assignee: North Health Essentials, LLC, Wilmington, DE (US)

(21) Appl. No.: 14/271,237

(22) Filed: May 6, 2014

Related U.S. Application Data
(60) Provisional application No. 61/863,831, filed on Aug. 8, 2013.

(51) Int. Cl. A61K 31/205; A23L 1/305; A23L 1/29; A61K 31/198; A61K 31/197

(52) U.S. Cl. CPC A61K 31/205; A61K 31/198; A61K 31/197; A23L 1/296; A23L 1/3051; A23V 2002/00
USPC 514/556

(57) ABSTRACT

A palatable, single serving dietary supplement of amino acids that may be taken as a nutritional aid to improve health and well-being by promoting higher quality sleep, enhancing repair of muscular tissue after strenuous exercise in part through boosting the natural production of human growth hormone, enhancing the immune system, improve gut barrier function, aiding in responses to upper respiratory viral pathogens, reversing side effects from the over use of nutritional energy drinks with stimulants and simple sugars and reverse the side effects of excess signaling through the cortisol pathway resulting from chronic environmental stress.

### CROSS REFERENCE TO RELATED APPLICATIONS

This application claims the benefit of U.S. Provisional Application No. 61/863,831 filed Aug. 8, 2013, the entire contents of which are incorporated herein by reference.

### FIELD OF THE INVENTION

The present invention relates to a palatable, single serving, liquid-formulated dietary supplement of several defined amino acids that promotes higher quality sleep, enhances repair of muscular tissue after strenuous exercise, strengthens the immune system, improves gut barrier function and provides relief from the onset of upper respiratory pathogens. This invention also relates to reversing side effects from the over use of so-called nutritional energy drinks and the adverse side effects of excess signaling through the cortisol pathway resulting from chronic environmental stress. This is defined as “The amino acid supplement effect” or TAASE and is shown in FIG. 1.

### BACKGROUND OF THE INVENTION

The past decade has seen an increasing consumption of nutritional energy drinks that usually contain relatively high levels of caffeine, related stimulants and simple sugars. The habitual use of these beverages is considered safe within some suggested limits by the US FDA. However, they may cause consumers to suffer from various stages of adrenal exhaustion, sleep deprivation and chronic inflammatory complaints due to poor recovery. Note these conclusions may be anti-intuitive since high levels of cortisol are associated with immunosuppression. However, the levels of cortisol seen in stress-related disorders are not at the levels seen in immunoregulatory responses that function to blunt responses to over-active immune responses seen during acute infections.

Separately, modern societies have moved to larger amounts of shift work and rotating 24 hour schedules. Many of these laborers have demonstrated chronic sleep deficits due to an inability to sleep during a rotating sleep period or in their ability to maintain a restful sleep cycle.

This invention was designed with the three-fold purpose of promoting (1) restful sleep and (2) supplying amino acids associated with tissue repair by, in part, promoting the secretion of human growth hormone and reducing inflammation (3) and enhancing immune responses to viral infections due to upper respiratory pathogens.

Notably, and in contrast to many other sleep aids, the active components of this formulation are only amino acids and the formulation does not contain other commonly used over-the-counter sleep aids such as melatonin, L-tryptophan or 5-hydroxytryptophan, valerian extracts, chamomile or chamomile extracts and diphenylhydramine. Tryptophan is not included in this formulation to increase safety in humans who may have been prescribed one of a class of drugs termed SSRIs or selective serotonin-reuptake inhibitors. That is due to the fact that tryptophan is readily metabolized in the body to serotonin. For this reason tryptophan and 5-hydroxytryptophan are not recommended for individuals taking prescription SSRI drugs or monoamine oxygenase inhibitors, known as the MAOIs.

The invention does not include the use of melatonin since there is a growing concern that its use of may lead to a diminished production of naturally occurring melatonin and may cause a dependency to develop. In addition to the above listed reasons, these compounds were left out because they treat the symptoms associated with sleep difficulties instead of targeting the underlying metabolic causes.

### SUMMARY OF THE INVENTION

TAASE is promoted by providing nutritionally significant amounts of high grade amino acids L-arginine, L-lysine, gamma-aminobutyric acid or “GABA,” L-glutamine, and glycine. Also included are the cyclic lactam L-pyroglutamate to enhance L-arginine utilization and the amino acid neurotransmitter gamma-aminobutyric acid. An antioxidant such as sodium sulfite or a phenolic compound may be included. A suitable anti-microbial preservative such as potassium sorbate or potassium benzoate may also be added. Disodium inosinate may also be added to mask the poor taste of some of the amino acids. Taken together, the ingredients have been shown to help improve sleep quality and quantity.

TAASE is also promoted by providing these amino acids in nutritionally significant amounts and in a palatable liquid formulation that is easy to consume. It can also be made in a dry formulation using a suitable dispersant such as citric acid and sodium or potassium bicarbonate which is a simple mixture of a weak acid and a weak base. The reaction between the weak acid and sodium or potassium bicarbonate causes the formation of carbon dioxide and disbursement of the amino acids.

It was found that less than all of the ingredients display efficacy in promoting sleep and recovery. These combinations include five combinations where one of the above listed five amino acids is not used and nine combinations were two of the five amino acids are not used. One of the combinations of three, namely the combination of L-arginine or L-arginine pyroglutamate, L-lysine and GABA did not have any noticeable effect. Empirical testing showed these fourteen combinations to be somewhat effective compared to the full formula and are thus part of the invention. Each of the fourteen combinations have either one or the other of, or both of, L-glutamine and glycine. However, the five ingredient combination is the most effective and therefore a preferred embodiment.

An object of the present invention is to provide these ingredients in a convenient liquid formulation.

A further aspect of the present invention is to provide these ingredients in a flavored palatable formulation.

Another object of the present invention is to provide these ingredients in a convenient single serving.

A further object of the present invention is to provide a group of amino acids in a formulation that promotes falling asleep faster and staying asleep longer.

Another object of the present invention is to shorten periods of nighttime wakefulness and promote falling back to sleep after waking during the night.

Another aspect of the invention helps increase the amount of Delta Wave sleep as well as REM sleep or Stage 3 and Stage 4 sleep.

A further object of the present invention is to provide a formula to help people recover from the adverse effects of stress.

Another object of the present invention is to provide a formula to enhance repair of muscular tissue after strenuous exercise or strenuous athletic competition.

A further object of the present invention is to provide a formula to promote quicker recovery after a surgical procedure.

Another object of the present invention is to provide a formula to strengthen the immune system at the onset of the common cold and complicating illnesses such as bronchitis to avoid the full manifestations of those illnesses.

A further aspect, this invention provides a method of improving immune function as well as gut barrier function.

Another object of the present invention is to provide a formulation that reduces morning grogginess after a full nights' sleep.

The invention also provides a formulation that that does not promote physical dependency.

The invention may include an antioxidant, a zero glycemic index sweetener and an acidifying agent for lowering the ph.

Clearly, elements in accordance with the present invention may be embodied in a wide variety of forms, some of which may be quite different from those of the disclosed embodiments. Consequently, the specific details disclosed herein are merely representative; yet in that regard, they are deemed to afford a preferred embodiment for purposes of disclosure and provide a basis for the claims herein which define the scope of the present invention.

### BRIEF DESCRIPTION OF THE DRAWINGS

The features of the invention will be more readily apparent from the following detailed description and drawings of an illustrative embodiment of the invention.

FIG. 1 is a table showing TAASE and the amino acids associated with the effects;

FIG. 2 is the structure of L-arginine pyroglutamate;

FIG. 3 is the structure of L-lysine;

FIG. 4 is the structure of GABA;

FIG. 5 is the structure of L-glutamine; and

FIG. 6 is the structure of glycine.

### DETAILED DESCRIPTION OF THE PREFERRED EMBODIMENT

Consistent with the present invention, a combination is disclosed for TAASE as follows: 126 ml of purified water or distilled water of low ionic strength and TDS (total dissolved solids) of around 15 to 20 ppm.; 2 grams of L-arginine pyroglutamate; 2 grams of L-lysine; 2 grams of GABA; 5 grams of L-glutamine and 3 grams of glycine. All are pharmaceutical grade obtained from NutraBio.com (Inc.). Also included are 1.2 grams of SweetenFX; 1.5 grams of citric acid; 0.0117 grams of the concentrated organic lemon flavoring all from Nature's Flavors (Orange, Calif.) and preservatives such as potassium sorbate and potassium benzoate. Disodium inosinate can be added to mask the unpleasant taste of some of the amino acids of the present invention if other flavorings are not added. In a preferred embodiment, the above amino acids are non-sodium containing high grade. Based on empirical testing, it is believed that high grade amino acids are more effective than food grade amino acids.

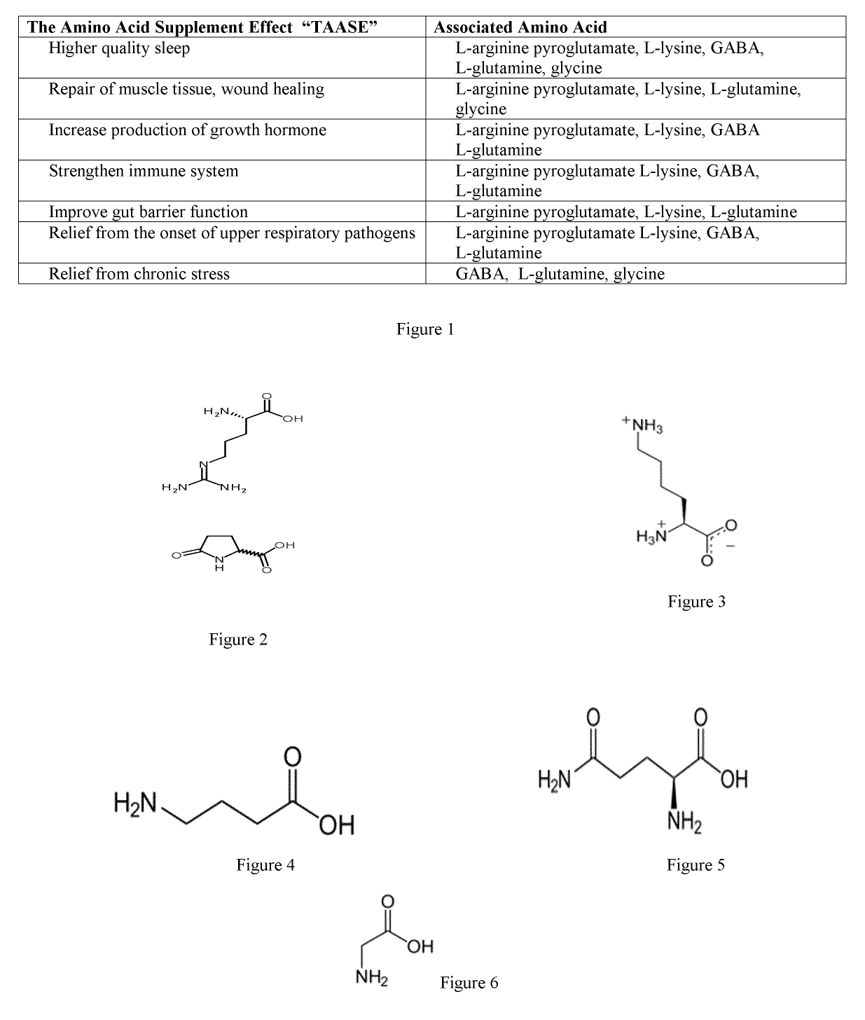

FIG. 1 is a table showing TAASE and the amino acids associated with each effect. It is contemplated that the present invention may be useful in a number of healing arts that are not listed in FIG. 1. FIGS. 2 through 6 show the structures for L-arginine pyroglutamate, L-lysine, GABA, L-glutamine and glycine, respectively.

L-Arginine Pyroglutamate. When consumed in combination with L-lysine, L-arginine has been shown to increase growth hormone secretion. One reason this combination might be so effective is that free form “L” versions of lysine and arginine compete for the same receptors. Replacing L-arginine with L-arginine pyroglutamate prevents competition for the same receptors. It is suggested that L-arginine pyroglutamate utilizes a different receptor allowing for a synergistic effect between the two amino acids. L-arginine can be used, however L-arginine pyroglutamate is preferred. The amino acids L-glutamine and L-arginine have been shown to improve both gut barrier function and immune function. Some people use L-arginine for preventing the common cold, improving kidney function after a kidney transplant, high blood pressure during pregnancy (pre-eclampsia), improving athletic performance, boosting the immune system and preventing inflammation of the digestive tract in premature infants. http://www.nlm.nih.gov/medlineplus/druginfo/natural/875.html

L-Arginine has also been shown to improve recovery and wound healing. Some studies have shown that increased production of nitric oxide in the forebrain is needed to produce sleep. It is believed that because L-arginine is converted into nitric oxide that it can help improve the production of nitric oxide in the forebrain thus helping induce a state of sleepiness.

L-Lysine. When combined with L-arginine pyroglutamate, L-lysine has been shown in studies to increase growth hormone secretion using a much smaller serving size than just using L-arginine alone. L-lysine decreases recovery times and helps the person get into a deeper level of sleep. L-lysine also improves the immune function and the gut function barrier. It is believed that L-lysine can help in providing relief from the onset of upper respiratory pathogens. A University of Maryland publication states that, “L-lysine”, or lysine, is an essential amino acid. That means it is necessary for human health, but the human body can't manufacture it. The human body must get lysine from food or supplements. Amino acids like lysine are the building blocks of protein. Lysine is important for proper growth, and it plays an essential role in the production of carnitine, a nutrient responsible for converting fatty acids into energy and helping to lower cholesterol. Lysine appears to help the body absorb calcium, and it plays an important role in the formation of collagen, a substance important for bones and connective tissues including skin, tendon, and cartilage. “ http://www.umm.edu/altmed/articles/lysine-000312.htm

Gamma-Aminobutyric Acid, (GABA). GABA is helpful because it induces relaxation, reduces anxiety, increases growth hormone production and improves sleep. “After 60 minutes of administration, GABA significantly increases alpha waves and decreases beta waves compared to water or L-threonine. These findings denote that GABA not only induces relaxation but also reduces anxiety.”

“In conclusion, GABA could work effectively as a natural relaxant and its effects could be seen within 1 hour of its administration to induce relaxation and diminish anxiety. Moreover, GABA administration could enhance immunity under stress conditions.” http://www.ncbi.nlm.nih.gov/pubmed/16971751. It is believed that GABA can help in providing relief from the onset of upper respiratory pathogens.

GABA is a close analog of glycine. The similarity between the structures and actions of glycine and GABA suggest that their “receptors” are similar, if not identical. For years, it has been known that progesterone and pregnenolone act on the GABA receptor, to reinforce the protective, inhibitory effects of GABA. Estrogen has the opposite effect, inhibiting GABA's action.http://raypeat.com/articles/articles/gelatin.shtml

L-Glutamine. Several studies have shown L-glutamine in amounts between 3 and 5 grams helps improve immune function, improve gut barrier function, increase growth hormone secretion and can promote improved recovery from injury or strenuous exercise. The University of Maryland Medical Center reports “Glutamine is important for removing excess ammonia (a common waste product in the body). It also helps immune system function (a) and appears to be needed for normal brain function and digestion.” And “Certain medical conditions, including injuries, surgery, infections, and prolonged stress, can lower glutamine levels, however. In these cases, taking a glutamine supplement may be helpful.”http://www.umm.edu/altmed/articles/glutamine-000307.htm.

The American Journal of Clinical Nutrition states that “ninety minutes after the glutamine administration load both plasma bicarbonate concentration and circulating plasma growth hormone concentration were elevated.” These findings demonstrate that a surprisingly small oral glutamine load is capable of elevating alkaline reserves as well as plasma growth hormone.” htt.://www.ajcn.org/content/61/5/1058.abstract. The US National Library of Medicine National Institutes of Health also stated, “It is concluded that an oral mixture of glycine, glutamine [sic] and niacin can enhance growth hormone (GH) secretion in healthy middle-aged and elderly subjects.” http://www.ncbi.nlm.nih.gov/pubmed/14609312

Additionally, it has been observed that the present invention is further enhanced by using between 6 and 10 g of L-glutamine instead of the 3 to 5 g of L-glutamine, disclosed above, especially after strenuous physical exercise or athletic competition. It is also believed that L-glutamine can help in providing relief from the onset of upper respiratory pathogens.

Glycine. It has been shown in several studies that the use of glycine can improve recovery, help individuals sleep longer and more deeply and promote wound healing and tumor inhibition.

One study showed that in individuals that had been experiencing continuous unsatisfactory sleep had improved sleep quality and efficacy when supplementing with 3 grams of glycine. This study also stated, “Thus, a bolus ingestion of glycine before bedtime seems to produce subjective and objective improvement of the sleep quality in a different way than traditional hypnotic drugs such as benzodiazepines.” It also stated that the use of glycine had lessened daytime sleepiness and the individuals in the study had experienced improved performance.

htt://onlinelibrary.wiley.com/doi/10.1111/j.14798425.2007.00262.x/abstract?systemMessage=Wiley+Online+Library+will+be+disrupted+24+March+from+10-14+GMT+%2806-10+EDT%29+for+essential+maintenance

The amino acid producer Ajinomoto found in their research on glycine that “Taking the supplement within an hour before going to bed, the researchers said the subjects showed brainwave patterns associated with non-REM sleep sooner and slept longer than those who did not take the amino acid” and “A study of the brains of rats revealed that glycine accumulates in the pineal gland, a part of the brain associated with the rhythms of waking and sleeping.” http://www.nutraingredients.com/Industry/Amino-acid-promotes-sleep-finds-Ajinomoto.

A generous supply of glycine/gelatin, against a balanced background of amino acids, has a great variety of anti-stress actions. Glycine is recognized as an “inhibitory” neurotransmitter, and promotes natural sleep. Used as a supplement, it has helped to promote recovery from strokes and seizures, and to improve learning and memory. But in every type of cell, it apparently has the same kind of quieting, protective anti-stress action. The range of injuries produced by an excess of tryptophan and serotonin seems to be prevented or corrected by a generous supply of glycine. Fibrosis, free radical damage, inflammation, cell death from ATP depletion or calcium overload, mitochondrial damage, diabetes, etc., can be prevented or alleviated by glycine.

The free fatty acids inhibit the oxidation of glucose for energy, creating insulin resistance, the condition that normally increases with aging, and that can lead to hyperglycemia and “diabetes.” Gelatin and glycine have recently been reported to facilitate the action of insulin in lowering blood sugar and alleviating diabetes.

A small dose of glycine taken shortly after suffering a stroke was found to accelerate recovery, preventing the spreading of injury through its inhibitory and anti-inflammatory actions. It's nerve-stabilizing action, increases the amount of stimulation required to activate nerves, is protective in epilepsy, too. This effect is important in the regulation of sleep, breathing and heart rhythm. http://raypeat.com/articles/articles/gelatin.shtml

Other ingredients. Other components are used to ensure the formulation is palatable. The ingredients include citric acid, SweetenFX, and the antimicrobial agents potassium sorbate and potassium benzoate.

An antioxidant may be added to scavenge for oxygen and improve shelf life.

Purified water is used because it has a TDS (total dissolved solids) of around 15 to 20 ppm. It was also discovered that a slightly more dilute solution of amino acids improves the ability to control flavoring.

SweetenFX is used as a sweetener because it has a zero glycemic index and does not cause a rise or fall in blood sugar which can cause a secretion of cortisol promoting wakefulness. The useful range is estimated to be between about 240 mg and 3 g per serving.

Citric acid is used to lower the pH below about 4.6. The useful range is estimated to be between about 360 mg and 4 g per serving.

Disodium inosinate can be added to mask the unpleasant taste of some of the amino acids of the present invention. It binds to receptors that would otherwise detect the unpleasant taste of some of the amino acids of the present invention.

Substances Not Included

Melatonin. The composition does not use melatonin because there is a growing concern that it's use of may lead to a diminished production of naturally occurring melatonin in the individual. This may cause a dependency on the hormone and increase the individual's difficulty falling asleep when it is not used. People might find that they have to keep increasing the dosage to get the same level of effectiveness from melatonin. However, even at higher dosages eventually it will begin to have a diminished effect.

L-Tryptophan or 5-Hydroxytryptophan. The invention does not use L-tryptophan or 5-hydroxytryptophan. These compounds are not recommended for individuals taking any SSRI drug, anti-depressant or MAOI inhibitor. L-Tryptophan and 5-HTP are metabolized directly into serotonin in the body. A person taking an SSRI, anti-depressant or MAOI inhibitor keeps serotonin cycling in the body longer. The use of L-tryptophan or 5-HTP and an SSRI, anti-depressant or MAOI inhibitor together may cause the person to suffer from Serotonin Syndrome, a potentially fatal neurotransmitter reaction.

The few studies that have been done indicate that the requirements for tryptophan and cysteine become very low in adulthood. Restricting only tryptophan, or only cysteine, produces a greater extension of the life span than achieved in most of the studies of caloric restriction. Since excess tryptophan is known to produce muscle pain, myositis, even muscular dystrophy, gelatin is an appropriate food for helping to correct those problems, simply because of its lack of tryptophan. (Again, the popular nutritional idea of amino acids as simply building blocks for tissues is exactly wrong--muscle protein can exacerbate muscle disease.) All of the conditions involving excess prolactin, serotonin, and cortisol (autism, postpartum and premenstrual problems, Cushing's disease, “diabetes,” impotence, etc.) should benefit from reduced consumption of tryptophan. But the specifically antiinflammatory amino acids in gelatin also antagonize the excitatory effects of the tryptophan-serotonin-estrogen- prolactin system. http://raypeat.com/articles/articles/gelatin.shtml

Other excluded substances include: 7keto DHEA; valerian extracts; chamomile or chamomile extracts and diphenylhydramine. These substances are inconsistent with the purpose of the invention because they treat the symptoms associated with sleep difficulties instead of targeting the underlying metabolic causes. Also, some of the above ingredients tend to cause morning grogginess which the present invention seeks to avoid.

The composition is made in liquid form because it is easy to use and the liquid formulation may aid in absorption. It is prepared by heating purified water to between approximately 120° to 200° F. and adding it to the mixing container. Then the flavoring and all the other ingredients are added to the mixing container. The ingredients do not dissolve well at room temperature given the large amount of dissolved solids in 126 mL of water. Citric acid is added to reduce the pH of the product to about 4.14.

The 9 three ingredient versions of the formula mentioned above are made in the same way except that some of the other ingredients such as citric acid, SweetenFX and flavoring are suitably adjusted to accommodate the lesser amount of active ingredients. The four ingredient formulas can also be made with the same type of adjustments. In both the three and four ingredient versions, the amount of water can be reduced because of the lesser amount of total dissolved solids. A three, four or five ingredient travel version can be made that has a total liquid volume of approximately three ounces and a two ounce version could be made to provide relief from the onset of upper respiratory pathogens.

The effective combination of active ingredients as stated above, 2 grams of L-arginine pyroglutamate; 2 grams of L-lysine; 2 grams of GABA; 5 grams of L-glutamine and 3 grams of glycine has a noticeably lowered efficacy at about 20% of the above amount based on empirical testing. This would be at least 400 mg of L-arginine or L-arginine pyroglutamate, at least 400 mg of L-lysine, at least 400 mg of GABA, at least 1000 /mg of L-glutamine and at least 600 mg of glycine. At 20%, of the preferred embodiment, the total amount of active ingredients is approximately 2800 mg. The three ingredient versions, with 3 of the following 5 ingredients, have a noticeably lowered efficacy at about 25% of the preferred embodiment or at least 500 mg of L-arginine or L-arginine pyroglutamate, at least 500 mg of L-lysine, at least 500 mg of GABA, at least 1250 mg of L-glutamine and at least 750 mg of glycine. The four ingredient versions have a noticeably lowered efficacy at about 20% of the preferred embodiment with 4 of the following 5 ingredients: at least 400 mg of L-arginine or L-arginine pyroglutamate, at least 400 mg of L-lysine, at least 400 mg of GABA, at least 1000 mg of L-glutamine and at least 600 mg of glycine.

The present invention can be made in multiple serving quantities of almost any amount. However, it was found during testing that approximately 20 g of dissolved solids was a maximum amount that could be dissolved in approximately 118 mL of water. At this concentration the dissolved solids started to precipitate out as the temperature fell below approximately 60° F.

The specific embodiments and examples set forth above are provided to illustrate the invention and are not intended as limiting. Additional embodiments within the scope of the claims will be apparent to those skilled in the art.

What is claimed is:

1. A composition comprising at least 400 mg of L-arginine or L-arginine pyroglutamate, at least 400 mg of L-lysine, at least 400 mg of GABA, at least 1000 mg of L-glutamine and at least 600 mg of glycine.

2. The composition of claim 1, wherein said composition comprises 2 g by weight of L-arginine pyroglutamate, 2 g by weight of L-lysine, 2 g by weight of GABA, 5 g by weight of L-glutamine, and 3 g by weight of glycine.

3. The composition of claim 1, wherein said composition is dissolved in water.

4. The composition of claim 1, wherein said composition further comprises SweetenFX in the range of 240 mg to 3 g.

5. The composition of claim 3, wherein said composition further comprises citric acid in the range of 250 mg to 4 g.

6. The composition of claim 5, wherein said composition further comprises a flavoring.

7. The composition of claim 6, wherein said flavoring is a lemon flavoring.

8. The composition of claim 1, wherein said composition further includes a weak acid and sodium or potassium bicarbonate as a dispersant.

9. A composition comprising any 4 of the following 5 amino acids:
at least 400 mg of L-arginine or L-arginine pyroglutamate, at least 400 mg of L-lysine, at least 400 mg of GABA, at least 1000 mg of L-glutamine and at least 600 mg of glycine.

10. A composition comprising any 3 of the following 5 amino acids, but excluding the combination of L-arginine or L-arginine pyroglutamate, L-lysine and GABA:
at least 500 mg of L-arginine or L-arginine pyroglutamate, at least 500 mg of L-lysine, at least 500 mg of GABA, at least 1250 mg of L-glutamine and at least 750 mg of glycine.

11. The composition of claim 9 or 10, wherein said composition is dissolved in water.

12. The composition of claim 11, wherein said composition further comprises SweetenFX in the range of 240 mg to 3 g by weight.

13. The composition of claim 12, wherein said composition further comprises citric acid in the range of 250 mg to 4 g.

14. The composition of claim 13, wherein said composition further comprises a flavoring.

15. The composition of claim 14, wherein said flavoring is a lemon flavoring.

16. The composition of claim 9 or 10, wherein said composition further includes a weak acid and sodium or potassium bicarbonate as a dispersant.

17. A method of making an aqueous solution of amino acids, comprising:
heating water with low dissolved solids to a temperature between 120 and 200° F.;
adding a flavoring to said water;
adding a group of amino acids and a sweetener to said water;
mixing said amino acids and sweetener until they are dissolved; and
adding acidifying agent to said water to bring the pH below 4.6.

18. The method of claim 17 wherein said flavoring is a lemon flavoring.

19. The method of claim 17 wherein said sweetener is SweetenFX.

20. The method of claim 17 wherein said acidifying agent is citric acid.
